(19) United States
(12) Patent Application Publication
(10) Pub. No.: US 2009/0299486 A1
(43) Pub. Date: Dec. 3, 2009

(54) Gastrointestinal Prostheses

(75) Inventors: Shaul Shohat, Kfar Hoaranim (IL); Raz Bar-On, Tel Aviv (IL)

Correspondence Address: Avia Kafri, 22 Shderot Chen St., Tel Aviv, 64166, IL

(73) Assignee: Slimedics Ltd., Kiryat Gat (IL)

(21) Appl. No.: 12/308,848

(22) PCT Filed: Jun. 28, 2007
(86) PCT No.: PCT/IL2007/000799

§ 371 Date: Dec. 24, 2008

Related U.S. Application Data
(60) Provisional application No. 60/817,071, filed on Jun. 29, 2006.

(51) Int. Cl. A61F 2/04; A61M 29/00

(52) U.S. Cl. 623/23.65; 606/192

(57) ABSTRACT

Gastrointestinal prosthesis for restraining the rate of the gastric digestion consists of a compressible proximal member connected by a string to a distal member, to be introduced into the intestine. The proximal member has an internal space, which is opened to the gastric lumen and may include passageways for the gastric content as well as open grooves or niches disposed on its external wall. The volume of a proximal member compressed by the gastric wall cannot get smaller than a lower threshold. Anchoring the gastrointestinal prosthesis at its desired location is accomplished by means of the proximal member the geometrical shape of which conforms the geometrical shape of a portion of the gastric lumen; as well as by means of the geometrical shape of the distal member which conforms the geometrical shape of a segment of the duodenum and/or a segment of the intestine.

### FIELD OF THE INVENTION

The present invention relates in general to methods and systems for treating obesity. More particularly the present invention relates to gastrointestinal prostheses providing for restraining gastric digestion.

### BACKGROUND OF THE INVENTION

Treating obesity often requires a surgical intervention. A lot of efforts have been invested to develop systems and methods which can be classified as being minimally invasive. In US patents U.S. Pat. No. 7,111,627S and U.S. Pat. No. 7,121,283 a system providing for weight loss including a tubular prosthesis to be introduced into a gastric lumen is disclosed. The prosthesis, which is self expandable, touches the gastric wall, thereby modulating the release of Gherin is accomplished. Therefore a feeling of satiation is induced.

In a US patent U.S. Pat. No. 6,994,095 a device occluding the distal gastric opening is disclosed. Such device provides for reducing the rate of flow of gastric content into the intestine, which in turn helps in reducing quantities of eaten food and extending the time intervals between meals.

In a US patent U.S. Pat. No. 7,220,284 a system including a number of components that can be used separately or in combination. The components of the disclosed system include prostheses that can be employed for reducing the volume of the stomach, by-passing a portion of the stomach and/or the small intestine. The disclosed system provides for reducing nutrient absorption and/or depositing minimally or undigested food into the intestines.

Any system and method, which may restrain the gastric digestion process, that will be more convenient to the surgeon, less painful to the patient, namely is not involved with obstructing passageways within the gastrointestinal system, and avoids attaching to the tissues engaged, such as by stitching, is beneficial.

### BRIEF DESCRIPTION OF THE DRAWINGS

FIG. 1 is a schematic description of a proximal member of a gastrointestinal prosthesis according to the present invention deployed within the stomach;

FIGS. 2a-3b schematically show proximal members according to five different embodiments of the present invention respectively;

FIG. 3c schematically presents a detail of the proximal member shown in FIG. 3b;

FIG. 3d schematically describes the proximal member shown in FIG. 3b emerging off a catheter;

FIG. 3e is an isometric view of a proximal member of a gastrointestinal prosthesis according to a preferred embodiment of the present invention;

FIG. 4a schematically shows an introducing system providing for introducing gastrointestinal prostheses of the invention into a gastrointestinal system;

FIG. 4b schematically shows another introducing system providing for introducing gastrointestinal prostheses of the invention into a gastrointestinal system;

FIG. 5a-5b respectively show two proximal members in accordance with two preferred embodiments of the invention placed within a gastric lumen respectively;

FIG. 6a-6c respectively show three proximal members in accordance with three different preferred embodiments of the present invention placed within a gastric lumen;

### DETAILED DESCRIPTION OF THE PRESENT INVENTION

In accordance with the present invention a system and method for restraining grinding and mashing of the content of the stomach is provided. The system of the invention consists of an intra gastric prosthesis providing for restraining the forces exerted on the digested food by flexing and traction of the stomach muscles. Therefore the time in which chyme, a homogeneous creamy or gruel-like material produced by gastric digestion, exits the stomach is significantly extended. The prostheses of the invention provide a space having a minimal volume in which foodstuff can not be smashed or grinded by the stomach. Such space is generated by one or more passageways through, and/or niches disposed on the surface of, the prostheses. The materials, such as foodstuff, contained within this space are exposed to a portion of the gastric lumen, which is external to the prosthesis. Chyme according to the present invention need not pass through a lumen of a prosthesis while being delivered towards the pylorus for exiting the stomach.

In accordance with the method of the present invention the gastrointestinal prostheses are not attached to any of the tissues engaged. Although the prostheses are free to move and/or rotate while being forced by the peristaltic motion of the organs involved they are anchored to, and do not migrate off, their targeted positions. Anchoring is accomplished by means of the geometrical shape of the components of prosthesis, which respectively conform to the geometry of the stomach, duodenum and/or the intestine. The gastrointestinal prostheses of the invention consist of a proximal member connected to a distal member, both are compressible. The proximal member is normally expanded such that a segment of its external surface is being at a close proximity to the inner surface of at least a portion of the stomach. The volume of the proximal member decreases when is compressed and forced by the gastric muscles down to a minimal threshold, which is larger compared to the volume of the respective space of a gastric lumen into which such prosthesis has not been introduced. The distal member whose geometrical shape conforms at least to a segment of the intestine extends into the lumen of the intestine.

All the materials utilized for manufacturing the components of gastrointestinal prostheses of the invention are biocompatible however none of them is degradable. In accordance with the present invention a prosthesis is taken off a patient either for the purpose of substituting with a new one, such as in cases in which a fault is detected, or following a planed decision of the physician who is responsible for the treatment.

### Proximal Member of a Gastric Prosthesis

Reference is now made to FIG. 1-3e in which various proximal members of gastric prostheses in accordance with different embodiments of the present invention are schematically shown respectively. In FIG. 1 proximal member 10 is disposed inwardly extending from pyloric antrum 12 into the lumen of stomach 14. Proximal member 10 consists of elastic wire 16 helically bended to form loops whose radii gradually increase conformal with the shape of the distal end of the gastric lumen. Pulling the proximal and distal ends of wire 16 in opposite directions opens the helical loops and respectively decreases their radii. Releasing both wire ends brings proximal member 10 back to its normal expanded configuration. The constants of elasticity of wire 16 are such that proximal member 10 is much more easily elongated rather than being laterally compressed. Therefore apart from being expanded to touch the inner surface of the stomach the loops resist and therefore dampen and restrain the contractions of the wall of a stomach. Namely, the forces exerted on pieces of food contained within the lumen of the proximal member are reduced due to the elastic forces exerted by the spring in the opposite directions. Furthermore, a significant portion of the energy transferred from the gastric muscles is turned into elastic energy of the proximal member. Most of this elastic energy is transferred back to the gastric wall during a respective expansion of proximal member 10, apart from a portion of the energy that is wasted on work against the compressing forces of the stomach's wall.

Ring 18 disposed at the distal end of wire 16 provides for connecting proximal member to the distal member of the gastric prosthesis, not shown, which is further described infra. A segment of wire 16, whose one end is the distal end of the wire passes through the lumen of pyloric sphincter 19.

The space enclosed within the internal surface of the proximal members, such as member 10, is regarded as an internal space of the respective proximal member. The volume of this space is regarded as the volume of the proximal member and is referred hereinafter as the volume of the proximal member. The elastic constants of proximal 10 are such that when is fully compressed by the gastric wall its value is not smaller than a predefined threshold. The value of this threshold, such as associated with proximal member 10 is significantly larger than the volume of a respective space of the gastric lumen when is similarly compressed and proximal member 10 is avoided. The body of a proximal member includes according to the present invention all the physical bodies constituting it including spaces enclosed within closed internal niches apart from the above mentioned space.

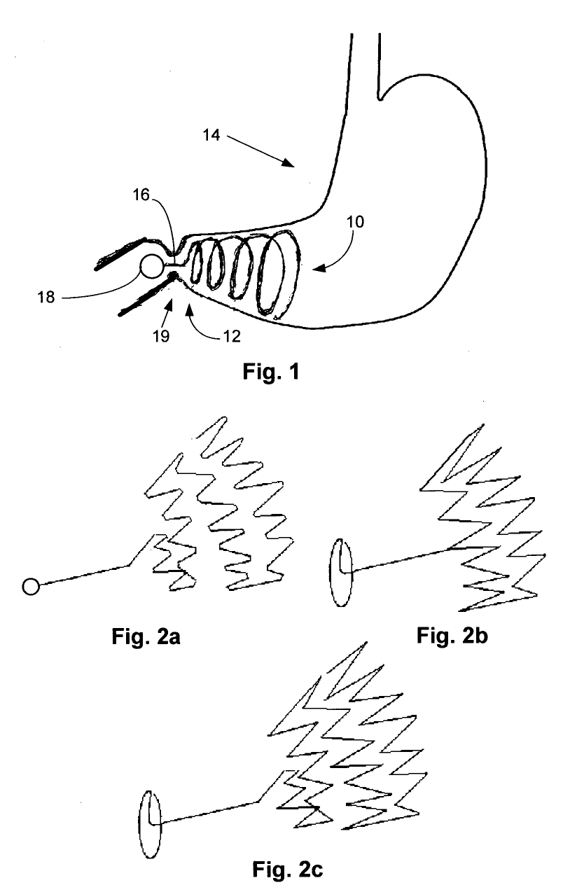

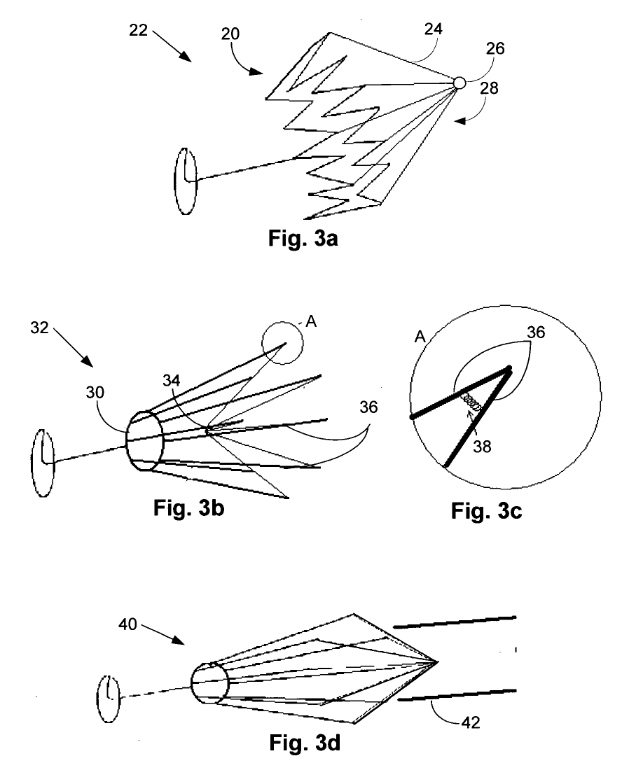

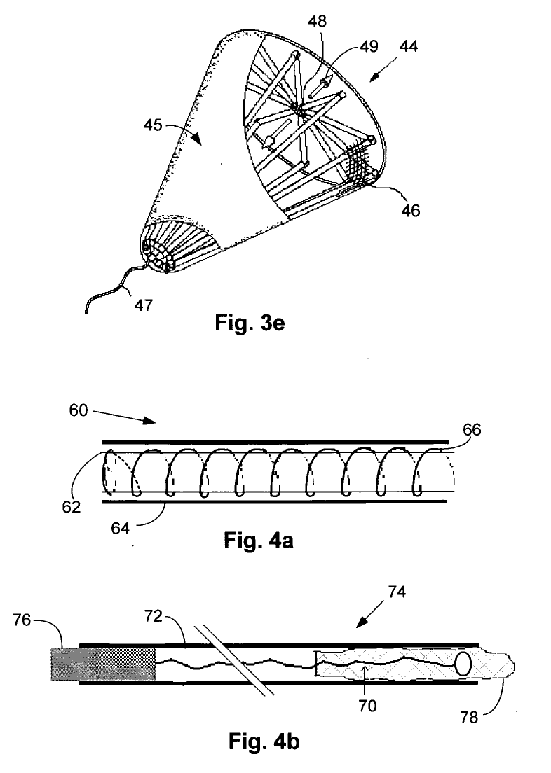

The helical loops of the proximal member of the gastric prosthesis shown in FIG. 2a have undulations whereas the wire from which the loop, or helical loops respectively shown in FIGS. 2b and 2c are bended such that they are shaped as the letter w. These bends and/or undulations provide for enhancing the structural strength in sustaining radial pressure such as exerted by the gastric wall. In FIGS. 3a-3e umbrella like proximal members are respectively shown. Inner loop 20 of proximal member 22 is repeatedly bended such that its adjacent segments are shaped like the letter “v”. Bars, such as bar 24 connects between corners of loop 20 and connecting hub 26 collectively structuring strengthening frame 28. Strengthening frame 28 provides for enhancing the resistance of proximal member 20 to pressure applied by the gastric wall. Inner loop 30 of proximal member 32 onto which v shaped pairs of pivotally attached bars, referred hereinafter as hinged bars and collectively designated by 36, are attached. The free end of one bar of a pair is firmly attached to loop 34 whereas all the free ends of the other bars of all the pairs are pivotally attached a hub disposed at a point displaced apart from loop 30. In order to enhance the resisting forces to pressure radially exerted on proximal member 32, elastic element 38, such as a spring, push against and/or pull together both arms of all the hinged bars respectively. Proximal member 32 is extended up to its normal span by the elastic restoring forces of the arms of the hinged bars whilst being stressed as well as by forces exerted by elastic elements 38. Furthermore the radius of loop 34 in addition to the elastic forces exerted by elastic elements 38 provides a minimal threshold to which the volume of the lumen of proximal member 32 can be decreased. Therefore the grinding and smashing of the foodstuff contained within the lumen of proximal member 32 by the stomach is significantly restrained. In FIG. 3d proximal member 40, which is the same as shown in FIGS. 3b and 3c is shown partially deployed while emerging off introducing catheter 42. In order to introduce proximal member 40 into an introducing catheter one has to pull proximally hub 43 thereby the hinged bars are pulled open and the maximal radius of proximal member 40 is thereby decreased.

In FIG. 3e an isometric view of a proximal member according to a preferred embodiment of the present invention is shown. Similarly to the proximal member described above with reference to FIG. 3b proximal member 44 has an inner loop onto which a structure consisting of hinged bars is attached. Conical cover 45 whose external layer is a sleeve made of fabric and net 46 interleaves between the sleeve and the hinged bars provides an enhanced mechanical strength to sustain lateral pressure. Optionally the conical sleeve and or the net can be avoided. String 47 provides for connecting proximal member 44 to a distal member. Following the placement and expanding of proximal member 44 at the targeted location, hub 48 is internally disposed within the lumen of proximal member 44. For introducing proximal member 44 into the gastrointestinal system it has first to be compacted and arranged for introducing. Compacting is accomplished by pulling out hub 48 in the direction of arrow 49. Hub 48 normally moves reciprocally along the direction of arrow 49 while the respective portion of the gastric lumen expands and in the opposite direction while proximal member 44 is forced inwards by the gastric walls .

Any metals or plastic resins which are normally utilized for manufacturing devices, tools and/or prostheses for the gastrointestinal system, except for materials which are biodegradable, can be utilized according to the invention for manufacturing proximal members of the gastrointestinal prostheses.

### Distal Member of a Gastric Prosthesis

The distal member of a gastrointestinal prosthesis provides according to the invention for anchoring the prosthesis in its targeted place within the gastrointestinal system. The distal member is free to distally and/or proximally move along the duodenum and/or intestine when is forced by the peristaltic motion and/or by being pulled as a result of forces exerted on the proximal member. The distal member cannot migrate off the intestine back to the gastric lumen due to its geometrical shape, which is conformal to the geometry of the intestine; and/or due to the geometrical shape of the proximal member, which is conformal to the geometry of the gastric lumen. Anchoring prostheses of the invention in place is not based on friction forces exerted by a surface or surfaces of the intestine onto the distal member. Anchoring is accomplished according to the present invention only by geometrical constraints preventing a linear displacement of any of the proximal or the distal members, which is longer than a predefined distance.

A distal member according to a preferred embodiment of the present invention is an inflatable balloon having three segments. The geometrical shape of the inflated balloon conforms the geometrical shape of the lumen of the duodenum, which is similar to the letter “C”. Preferable is a balloon whose geometrical shape consists three linear segment, namely its corners' angles are almost of 90° or it sides conform a piecewise linear line. The radius of the inflated balloon is significantly smaller compared to the inner radius of the duodenum. Therefore a clearance of a considerable volume provides for minimally interrupted digestion process within the lumen of the duodenum across all the segments of the balloon. However the lengths of the three segments respectively conform the lengths of the first three segments of the duodenum. Therefore the nearly linear sides of the inflated balloon cannot pass through the bends of the duodenum by a pull induced by the peristaltic movements or the proximal member. An inflating/deflating port provided with a valve is disposed at the proximal end of the balloon providing for connecting to an inflating canula as known. The balloon is connected to the proximal member of the gastric prosthesis by a filament, string or a wire of a predefined length. Optionally the balloon is elongated to have a fourth segment extending into the intestine distally to the ligament of Treitz. Three linear balloons serially connected may substitute the single balloon. Any flexible elongated spring, rod, wire, or string, extending towards the intestine and having a considerable length which may avoid pulling it off the duodenum, may serve as a distal member according to the invention. Balloons are preferable as they are easy to manipulate during the introduction and placement of the gastric prostheses of the invention. Any biocompatible material which is utilized for manufacturing balloons normally employed within the gastrointestinal system can be utilized for manufacturing a distal member of the invention.

### Introducing and Placing a Gastric Prosthesis

Introducing gastrointestinal prostheses of the invention into the gastrointestinal system of a patient is accomplished by means of gastric-introducing systems through the patient's mouth and the oesophagus, as known. Reference is now made to FIG. 4a in which a segment of a typical introducing system is schematically shown. Introducing system 60 has two concentric tubes, an inner tube 62 enveloped by an outer tube or introduction sheath 64. The inner tube provides for passing it along a guiding wire, delivering the balloons for their placement, introducing inflating canula and/or a trocar through, and/or introducing an imaging probe such as gastroscope. The space separating between the external sheath and the inner tube provides for passing through a prosthesis of the invention which is accordingly arranged. Such system is suitable for introducing gastrointestinal prostheses whose proximal members are hollow, such as those shown in FIGS. 1-2c, to which reference is again made. The helical loops are stretched open and the wire is wound across the surface of the inner tube as shown in FIG. 4a to which reference is again made. Proximal members such as shown in FIGS. 3a-3e, to which reference is again made, do not open to a single wire. For such gastrointestinal prostheses an introducing system as shown in FIG. 4b, to which reference is now made, is more suitable. Wire 70 is wiggled in different planes all of which cross at the axis of catheter 72 of introducing system 74. Trocar 76, or a pushing device provide for releasing the proximal member by pushing the proximal end of wire 70 along the lumen of the catheter towards its distal end. The distal end of wire 70 is enveloped with net 78 or with a sleeve such that when it is positioned in place within the pyloric antrum, the proximal member of the prosthesis is safely deployed when is spontaneously expanded such that hazards of injuries caused to the tissues engaged are minimized. Obviously net 78 is made of a biodegradable material. Alternatively proximal members are compressed as shown in FIG. 3d to which reference is again made, such that they are easily introduced into, and further be pushed and slide off the lumen of the catheter.

### EXAMPLE 1

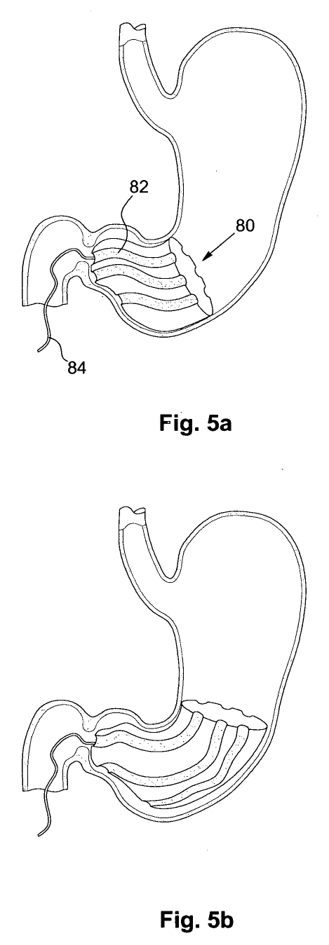

Three proximal members according to different preferred embodiments of the present invention are herby described with reference to FIGS. 5a-5c. Proximal member 80 is an inflatable balloon whose geometrical shape conforms the distal region of the gastric lumen within a close proximity the pyloric antrum. A plurality of grooves, such as groove 82, is disposed on the surface of the balloon. String 84 connects proximal member 80 to the distal member of the gastrointestinal prosthesis, not shown, to anchor it at its targeted place within stomach 86. Anchoring is accomplished by means of the geometrical shape of the distal member as described hereinabove.

The grooves are relatively narrow however their depth provides a considerable volume for containing partially digested foodstuff. The space enclosed within a surface of a groove and the external surface of the balloon in a case that such groove is avoided is regarded herein after a space internal to the balloon. The volume of the unified space including all the spaces of each groove of a balloon is referred hereinafter as the volume of the proximal member consisting of this ballon. The normal volume associated with the inflated balloon is defined at a state in which a considerable portion of the balloon's surface touches the gastric wall. In such a case a relatively narrow clearance is available between the surface of the balloon and the gastric wall. The volume of a compressed balloon decreases down to a lower volume, which is not smaller than a predefined threshold, when being repeatedly compressed by the gastric peristaltic motion. Typically the volume associated with the respective threshold is significantly larger compared to the volume of the respective portion of the gastric lumen when the balloon is avoided.

The level of stiffness of a proximal member consisting of such balloon can be adjusted by a relatively small volume of fluid injected into, or evacuated off, its lumen. The fluid is transferred through a valve associated with an inlet/outlet aperture of the balloon, not shown. Such adjustment provides for tuning the level stiffness, namely the level of resisting power exerted unto the gastric wall, as well as the level by which foodstuff is smashed or grinded by the gastric peristaltic motion. The width of the grooves is small enough such that the gastric wall cannot get into the grooves thereby significantly reduce their capacity. Therefore smashing or grinding foodstuff momentarily contained within a groove whilst being pressed by a gastric wall is significantly restrained. However any foodstuff contained within a groove is exposed to the fluids contained in the gastric lumen. Chyme delivered to the duodenum always exits the gastric lumen and need not be delivered through the grooves or any passageways internal to the proximal member if any such exists.

In FIGS. 5b similar proximal member is shown except that its volume is larger compared to the proximal member described above with reference to FIG. 5a.

### EXAMPLE 2

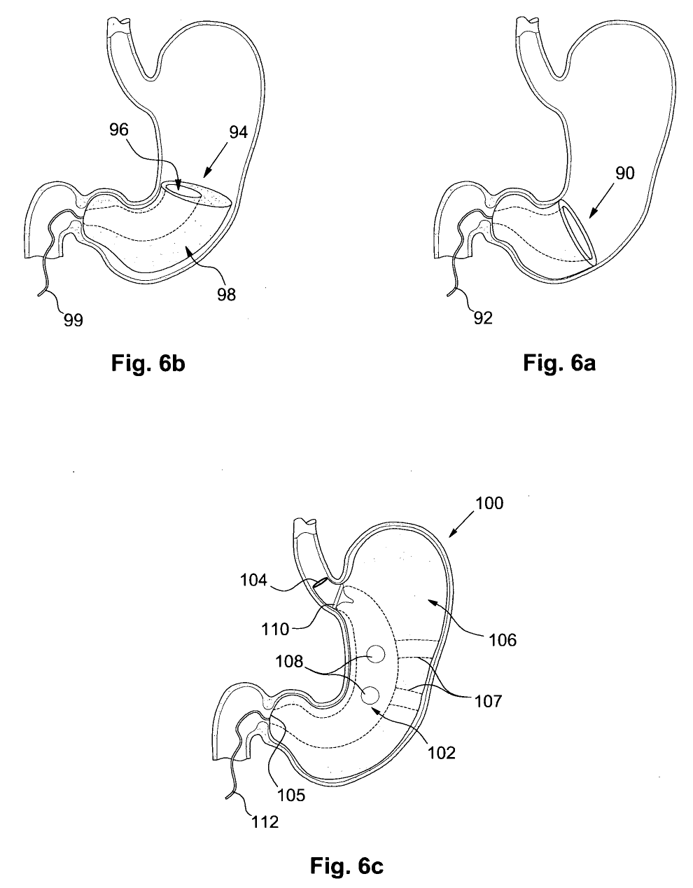

Reference is now made to FIGS. 6a-6c in which proximal members in accordance with three different preferred embodiments of the present invention are respectively shown. Al of the three proximal members shown are placed within the lumen of a stomach. All of them consist of an inflatable balloon having an internal passageway for delivering foodstuff and or chime through. The dimensions of proximal member 90 are accommodated to the lower region of the gastric lumen near the pyloric antrum. String 92 provides for connecting proximal member 90 to a distal member consisting of an inflatable balloon having a geometrical shape of the letter “C” when is inflated. The balloon constituting proximal member 90 is structured as a funnel whose small aperture is disposed within the pyloric antrum. At the proximal face of proximal member 90 another aperture is disposed. The wall of the funnel is hollow providing for a fluid, such as liquid and/or gas, to be injected and/or evacuated from its lumen. Liquids are preferable to gases since a tiny change in the quantity of contained liquid results in a significant change in the internal pressure. On top of the proximal wall of the balloon a three port aperture provided with a remote controlled valve having three states is disposed, not shown. One of these ports is provided with an attaching device providing for connecting with a canula for inflating/deflating the balloon. A second port is fluid connected to the lumen of the balloon, whereas the third port is open to the gastric lumen. While being in the first state the lumen of the balloon is closed, in the second state the lumen of the balloon is fluid connected with the first port, whereas in the third state the lumen of the balloon is connected with the third port, namely is open to the gastric lumen. The ballon is typically injected and pressurized with a fluid such as saline solution to which a dye is added, as known. Therefore leaking can be detected by checking the color of the patient's secretions. Optionally an additional compartment which is fluid isolated from the lumen of the balloon is attached to the proximal balloon's wall. A small remote controlled pump and a battery are also attached to the balloon. By pumping a relatively small quantity of liquid contained in the compartment and pressurizing it into the lumen of the balloon its stiffness and the resisting forces which can be exerted unto the gastric wall are increased and respectively decreased by evacuating some of the fluid.

Proximal member 94 is capable of occupying a portion of the gastric lumen which is larger compared to the respective portion that is occupied by proximal member 90. Proximal member 100 on the other hand is suitable for treating severe cases of morbid obesity. It occupies almost the entire space of the gastric lumen. Its main passageway 102 is opened to the esophagus through aperture 104 whose lumen forms continuity with the lumen of the esophagus. Another aperture of passageway 102 is aperture 105, which is opened to the pyloric antrum. Balloon body 106 interleaves between main passageway 102 and a portion of the gastric lumen. A number of passageways such as passageways 107 connect between the gastric lumen and main passageway 102. Such connections provide for transferring material between the main passageway and the gastric lumen, as well as for exposing the foodstuff contained in passageway 102 to gastric fluids. Apertures 108 are opened to passageways 107. The unified space consisting of the space enclosed within the main passageway together with the space enclosed within the other passageways is regarded as space which is internal to proximal member 100. The volume of this space is regarded as the volume of the respective proximal member.

Valve 110 is automatically closed when the inner pressure of the gastric lumen exceeds the pressure within the esophagus and opens when the respective pressure difference reverses. String 112 connects between proximal member 100 and the distal member, not shown. Optionally the distal member in this case is a string extending distally into the intestine. As the proximal member fills almost the entire space of the gastric lumen a risk of its getting off the esophagus and/or rotating such that its main passageway is off centered relative to the pyloric sphincter is minimal and therefore there is no practical need to use for example balloon instead of the string extending into the intestine.

A physician may select the distance between the proximal face and the distal face of a proximal member, the level of stiffness, the configuration of the proximal member and the associated volume threshold which is suitable to a patient prior to introducing a gastrointestinal prosthesis and positioning it in place. Following on the physician may adjust and modify the level of stiffness of the balloon and thereby modify the volume threshold associated with the respective proximal member, by means of the remote controlled pump without a need to operate the pump in situ. Furthermore in a case that a leak of the inflating fluid is detected the physician may evacuate all the fluids contained in the faulty balloon and further take the prosthesis off to be substituted with a new one. Such evacuation may be accomplished by means of the above mentioned remote controlled pump and three state valve.

1. A gastrointestinal prosthesis comprising
a compressible proximal member for restraining and dampening compressive forces exerted by a gastric wall;
a distal member connected to said proximal member, and wherein the geometrical shape of a segment of said proximal member conforms to the geometrical shape of a portion of a lumen enclosed within said gastric wall, which is the gastric lumen, and wherein a space internal to said proximal member is opened to said gastric lumen, and wherein the volume of said space is not smaller than a predefined threshold.

2. A gastrointestinal prosthesis as in claim 1, wherein said distal member comprises a body having a geometrical shape conformal with the geometrical shape of a segment of a human duodenum.

3. A gastrointestinal prosthesis as in claim 2, wherein said distal member comprises any item selected from a group of items consisting of a string, wire, spring, elastic rod, elastic wire, net, sleeve, inflatable balloon and any combination thereof.

4. A gastrointestinal prosthesis as in claim 1, wherein said proximal member comprises any item selected from a group of items consisting of a spring, elastic rod, elastic wire, net, sleeve, cover, inflatable balloon and any combination thereof.

5. A gastrointestinal prosthesis as in claim 4, wherein said balloon comprises at least one passageway, which is a first passageway, opened to said gastric lumen.

6. A gastrointestinal prosthesis as in claim 5, wherein said balloon further comprises at least one additional passageway, which is the second passageway, opened to said gastric lumen, and wherein said balloon further comprises an aperture, and wherein the lumen of said aperture forms a continuum with the lumen of said first passageway and the lumen of said second passageway.

7. A gastrointestinal prosthesis as in claim 6, wherein said first passageway has an aperture having a circumferential margin conformal with the lumen of the esophagus.

8. A gastrointestinal prosthesis as in claim 7, wherein a valve is distally disposed to said aperture.

9. A gastrointestinal prosthesis as in claim 3, wherein said inflated balloon contains a fluid.

10. A gastrointestinal prosthesis as in claim 4, wherein said inflated balloon contains a fluid.

11. A gastrointestinal prosthesis as in claim 9, wherein said fluid is a liquid.

12. A gastrointestinal prosthesis as in claim 10, wherein said fluid is a liquid.

13. A gastrointestinal prosthesis as in claim 10, wherein said proximal member further comprises a pump for pressurizing said fluid into said inflatable balloon.

14. A gastrointestinal prosthesis as in claim 13, wherein said proximal member further comprises a remote controlled valve.

15. A gastrointestinal prosthesis as in claim 13, wherein said pump is remote controlled.

16. A method for restraining the rate of a gastric digestion of a human by enclosing a space internal to a portion of a gastric lumen of said human within a compressible body, wherein the volume of said space is not smaller than a predefined threshold while said body is compressed by a gastric wall of said human, and wherein said space is opened to a portion of said gastric lumen, which is external to said space, and wherein the geometrical shape of a segment of the surface of said body conforms the geometrical shape of a portion of said gastric lumen while said body is expanded.

17. A method as in claim 16, further comprising securing said compressible body in place by connecting a distal member to said body, wherein said distal member is introduced into a lumen of a segment of the intestine of said human, and wherein a segment of said distal member conforms a segment of any of the organs selected from a group of organs consisting of the duodenum and small bowel of said human.
